(19) United States
(12) Patent Application Publication
(10) Pub. No.: US 2004/0238007 A1
(43) Pub. Date: Dec. 2, 2004

(54) Method and apparatus for decontaminating non-ambulatory victims or objects

(76) Inventors: Larry M. Jones, Boise, ID (US); Clifton J. Seusy, Boise, ID (US); Timothy W. Rhodes, Boise, ID (US)

Correspondence Address: PEDERSEN & COMPANY, PLLC, P.O. BOX 2666, BOISE, ID, 83701, US

(21) Appl. No.: 10/454,097

(22) Filed: Jun. 2, 2003

(51) Int. Cl. B08B 3/04

(52) U.S. Cl. 134/21; 134/32; 134/72; 134/99.1; 134/110; 134/133; 134/200

(57) ABSTRACT

A decontamination system for victims of chemical, biological, radiological, or other harmful exposure. The system effectively isolates a contaminated patient during a gross washing process and provides the highest level of protection to unit operators that may be dressed in street clothes. The system comprises an isolation apparatus, a washing chamber, an output enclosure, a method of passing a non-ambulatory patient through the system and a waste collection system. The parts of the system may function together as a single, integral unit for mass decontamination at the site of exposure. Further, the system may be used for non-ambulatory and ambulatory humans and/or animals and possibly equipment and other objects.

### BACKGROUND OF THE INVENTION

1. Field of the Invention.

This invention relates generally to decontamination means for use in the event of chemical, biological, or radiological attacks and/or accidents. More specifically, this invention provides a mobile decontamination system and method for cleansing non-ambulatory, human victims of chemical, biological, or radiological exposure. Alternatively, the system may be used to decontaminate objects or animals.

2. Related Art.

Occasional, accidental exposure to harmful chemical, biological, and radiological substances is an unfortunate consequence of continued study in these areas. In the event of contact with such substances, there is a need to immediately, efficiently and completely decontaminate both human victims and equipment. A number of methods have been developed for use in such incidents. These devices and procedures are meant to quickly cleanse ambulatory patients and/or prevent the spread of contamination. However, many such devices are not designed for on-site use or are incapable of handling non-ambulatory victims. There is a need for on-site decontamination means which are suitable for unconscious, or otherwise non-ambulatory, patients.

Portable shower units with collapsible walls such as those disclosed in U.S. Pat. No. 4,989,279 (Powell) and U.S. Pat. No. 5,544,369 (Roberts) have been developed for use outdoors. However, these units are poorly suited to mass decontamination procedures and are not compatible with non-ambulatory victims of exposure to hazardous substances.

Mobile isolation chambers have been developed to prevent further spread of hazardous substances in exposed areas. For example, U.S. Pat. No. 4,883,512 (Griffis) discloses a lightweight isolation chamber with an attached air filtration unit. The air filtration unit maintains a negative air pressure within the chamber. This allows clean air to enter the stall while asbestos particles are removed from the exhausted air. U.S. Pat. No. 5,061,235 (Hogan) provides an isolation tent which may be mounted atop a super-absorbent stretcher to transport emergency victims away from the scene of an accident. This tent isolates the airspace adjacent to and surrounding the body of the victim during transport. However, it does not provide for decontamination of the victim in the event of exposure to harmful agents.

Transportable decontamination units such as those disclosed in U.S. Pat. No. 4,858,256 (Shankman) and U.S. Pat. No. 5,607,652 (Hellmuth, et al.) have been developed for use in areas which have been subjected to chemical or biological exposure. Shankman discloses a self-contained decontamination unit for use by ambulatory personnel deployed at the site of a major chemical disaster. The invention comprises a multi-chambered vehicle for decontaminating disaster response personnel and their equipment. A first and second compartment combine to receive and cleanse personnel while a third area is dedicated to the decontamination of equipment which may then be put back into service. Waste from this process is contained within storage tanks for proper disposal. Hellmuth, et al. provide a chemical and biological decontamination chamber which may be transported to the scene of an accident. Once the unit is in place, contaminated items may be placed into a plurality of baskets which are situated atop a conveyor track passing through the chamber. When the conveyor is set in motion, the baskets are served into the chamber. Within the decontamination chamber, a mixture of hot air and steam penetrates the articles and is drawn off and exhausted to remove biological or chemical contaminants. This system, however, is inappropriate for human use.

The object of the present invention is to provide a decontamination method and apparatus for non-ambulatory victims of chemical, biological, radiological, or other harmful exposure. In addition, the apparatus is to be transportable so that it may be used in or near a hazard site. Other objects of the present invention will become clear through the accompanying description.

### SUMMARY OF THE INVENTION

The present invention is a decontamination system for victims, preferably including non-ambulatory victims, of chemical, biological, radiological, or other harmful exposure, and a method of doing the same. For convenience, the invented decontamination unit may be easily transported to a site proximal to the exposure. Preferably, the component parts of the decontamination system cooperate to provide a single, integral unit for mass decontamination. Further, the invented system may be operated primarily by workers in street clothes rather than workers clad in personal protective equipment (PPE) such as, for example, polypropylene suits. This factor significantly improves operator mobility and eliminates many of the restrictions that exist for workers in PPE suits.

The invented system preferably comprises an isolation apparatus, a washing chamber and means for passing a non-ambulatory patient through the space of the isolation apparatus and chamber. The preferred isolation apparatus and washing chamber cooperate to permit passage of rescue personnel, and victims in their care, between the hazard site and the wash unit without further spreading contaminants. Typically, appropriately protected (i.e. with PPE) members of hazardous material (HAZMAT) response teams are responsible for transporting non-ambulatory patients through the isolation apparatus from the site of exposure to the wash unit. Victims are received into the chamber via a receiving hatch. Preferably, stretchered patients are placed upon a conveyor track passing through the chamber. Within the washing chamber, harmful agents are neutralized and/or removed from the patient. Operators outside the unit may control the distribution of metered cleansing solutions by foot-pedal-activated spray nozzles. Scrubbing, hair removal, clothing removal, and/or other operations may also be performed by the unit operators. Access to the patient within the enclosure is provided via a plurality of sealed ports in communication with the chamber interior.

Contamination of the environment surrounding the decontamination system apparatus is prevented through various means. For example, in a preferred system, an air filtration system maintains a negative air pressure within the washing chamber and air being pumped out of the chamber is first filtered for chemical, biological, radiological, or other harmful substances. In addition, liquid and solid waste is generally collected in catch-tanks and bags for appropriate disposal.

### BRIEF DESCRIPTION OF THE DRAWINGS

FIG. 1 is a side cross-sectional view of a preferred embodiment of the decontamination system showing cooperation between the isolation apparatus and the washing chamber.

FIG. 2 is an end cross-sectional view of the washing chamber of FIG. 1 showing the basic form of the chamber and its interior.

FIG. 3 is side view of the washing chamber of FIG. 1 showing the positioning of the operable elements.

FIG. 4 is a top view of the washing chamber of FIG. 1 showing handling of a patient through glove-access ports and the reaching radius of operators using these glove ports.

FIG. 5 is an end cross-sectional view of the washing chamber of FIG. 1 demonstrating a possible operation of the decontamination system.

FIG. 6 is a side cross-sectional view of the preferred decontamination system demonstrating the passage of a patient through the washing chamber.

FIG. 7 is an end view of a patient situated atop an embodiment of a patient carrier for use in the decontamination system of FIG. 1 demonstrating the passage of fluids through holes in the patient carrier.

FIG. 8 is a schematic of the preferred cleansing solution supply and control system for use in the decontamination system of FIG. 1.

FIG. 9 is a schematic of the preferred liquid waste disposal system for use in the decontamination system of FIG. 1.

FIG. 10 is a side view of the washing chamber of FIG. 1 illustrating an embodiment of a collapsible leg assembly and the placement of various equipment and supplies within the chamber for storage or transport.

FIG. 11a is a cross-sectional view of a glove port for use in the washing chamber of FIG. 1 adapted to receive a quick-change glove assembly.

FIG. 11b is an elevation view of a quick change glove assembly for use with the glove port of FIG. 11a.

FIG. 12a is a cross-sectional view of an alternative glove port for use in the wash chamber of FIG. 1.

FIG. 12b is an elevation view of a quick-change glove assembly for use with the glove port of FIG. 12a.

### DESCRIPTION OF THE PREFERRED EMBODIMENTS

Referring to the figures, the decontamination system comprises an apparatus and method for decontaminating victims of exposure to chemical, biological, radiological or other harmful agents. The decontamination system is preferably portable to provide for on-site decontamination of emergency victims with minimal assistance from members of hazardous-material (HAZMAT) emergency response teams. A unique advantage of the decontamination system is its compatibility with unconscious and/or non-ambulatory victims. The system is designed to isolate a contaminated, non-ambulatory patient during a gross decontamination washing process while providing the highest level of protection to unit operators who may be dressed in street clothes.

Preferably, the decontamination system 1 comprises means for isolating contaminated victims during transport and a washing chamber where harmful agents may be effectively neutralized and/or removed from victims. The isolation apparatus 10 provides means of evacuation from the site immediate to chemical, biological, radiological or other exposure. After evacuation from the immediate exposure site, victims may be treated in the washing chamber 20 to minimize or eliminate complications inherent in accidental exposure situations. Thorough washing of the patient within this enclosure also prevents the further spread of contamination.

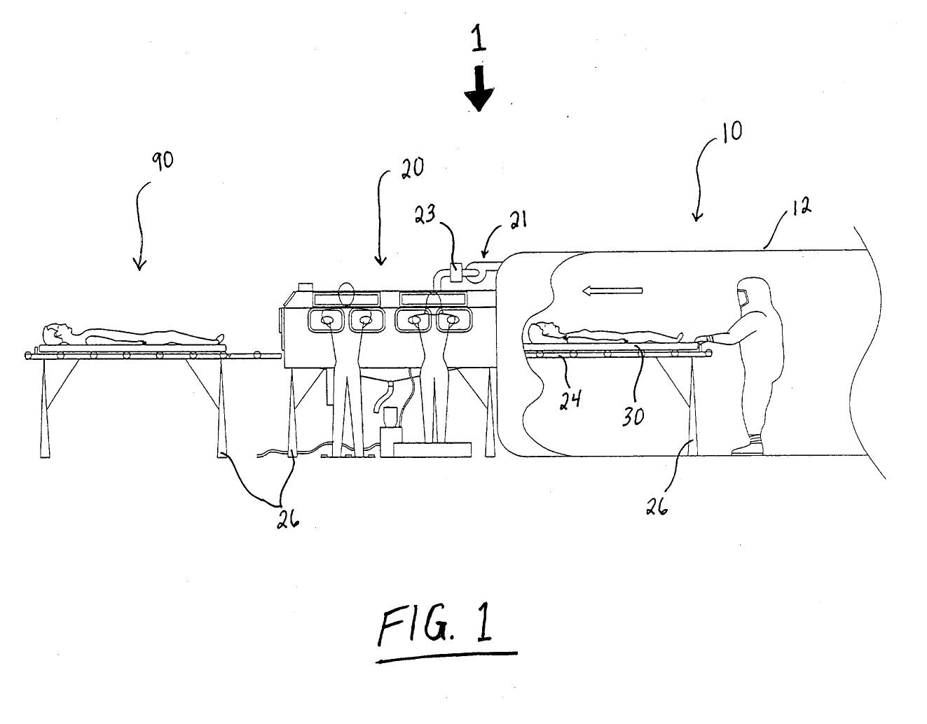

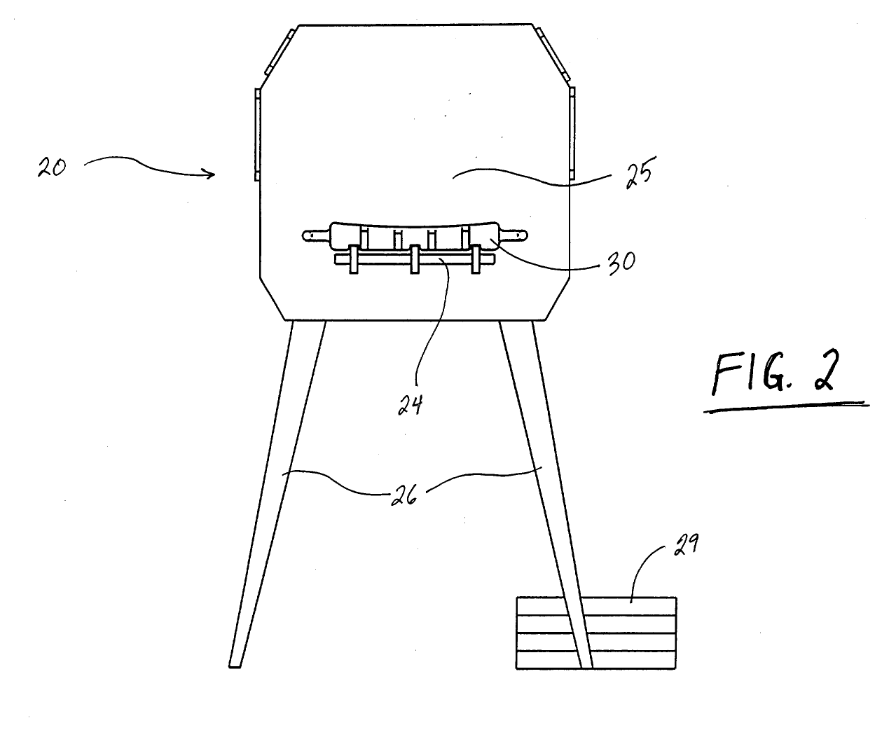

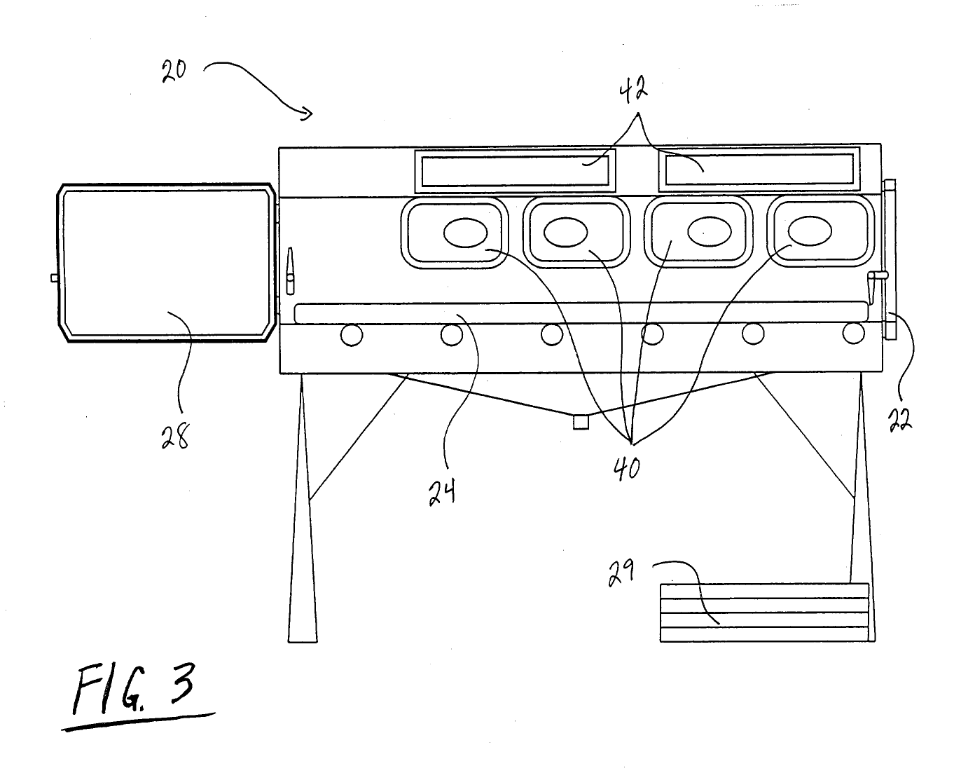

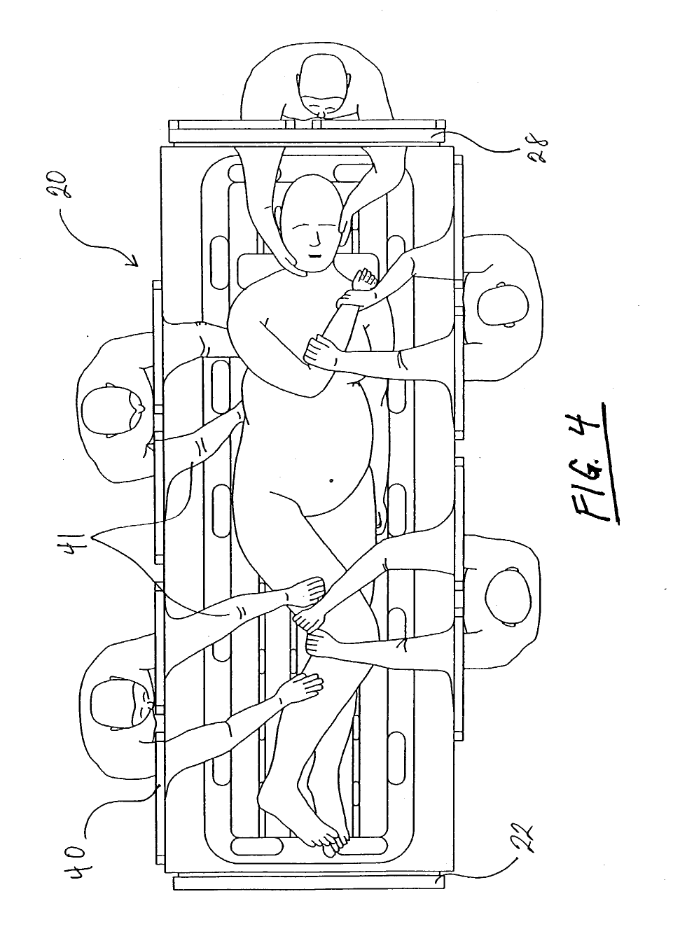

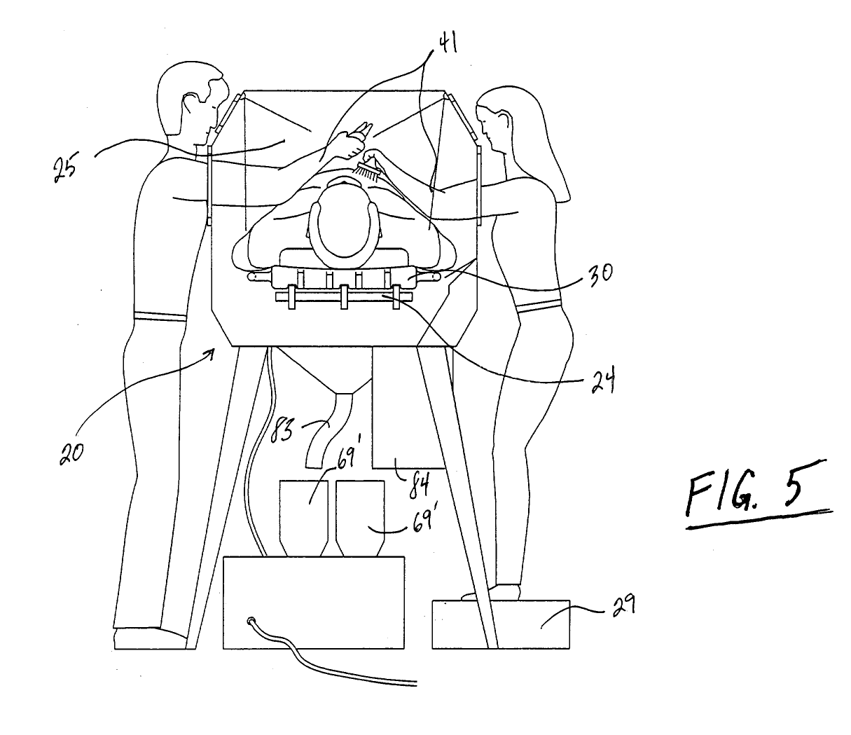

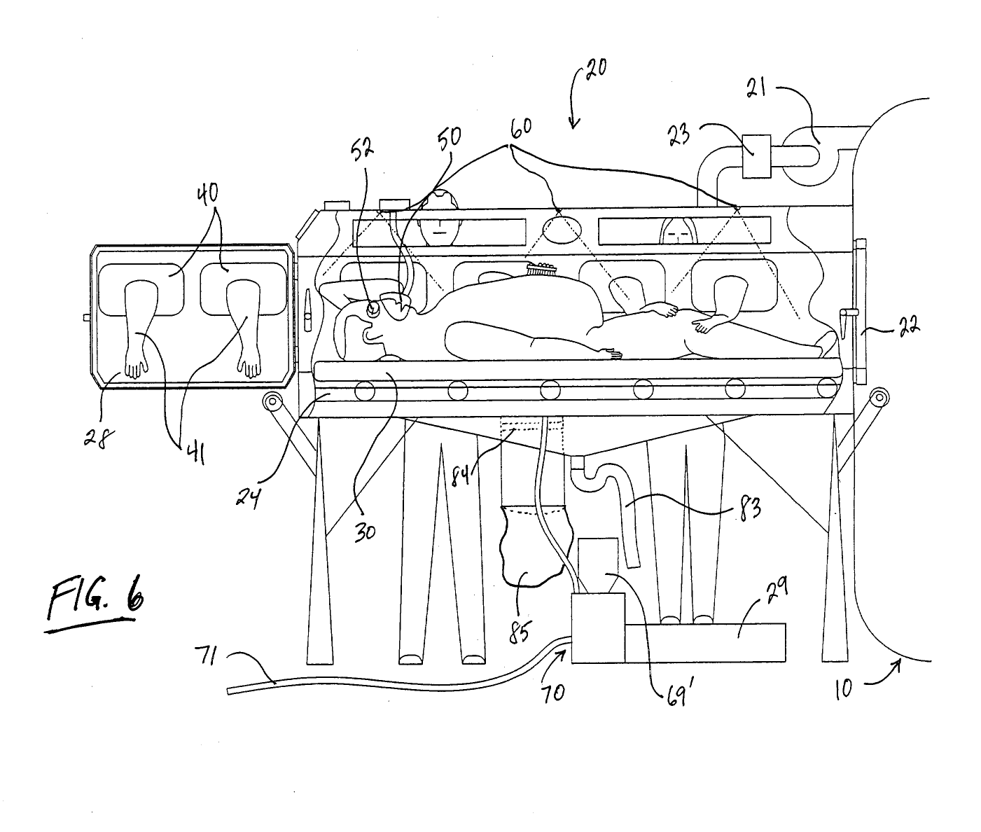

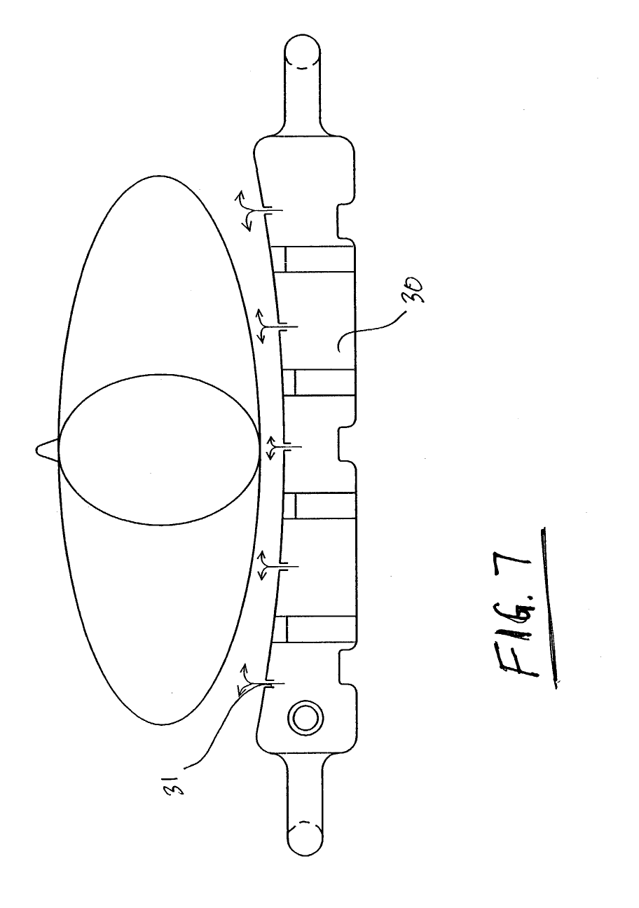

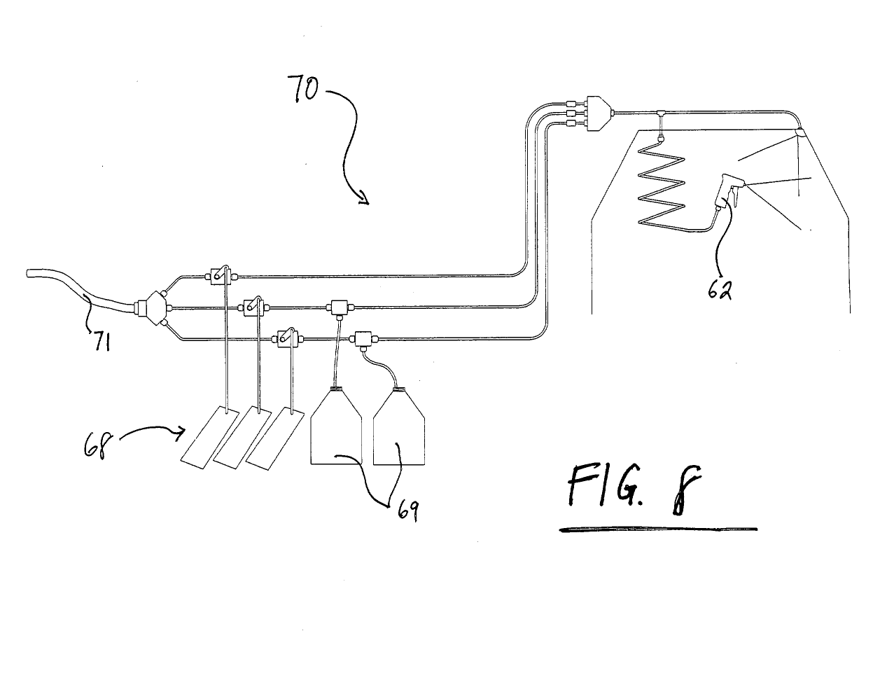

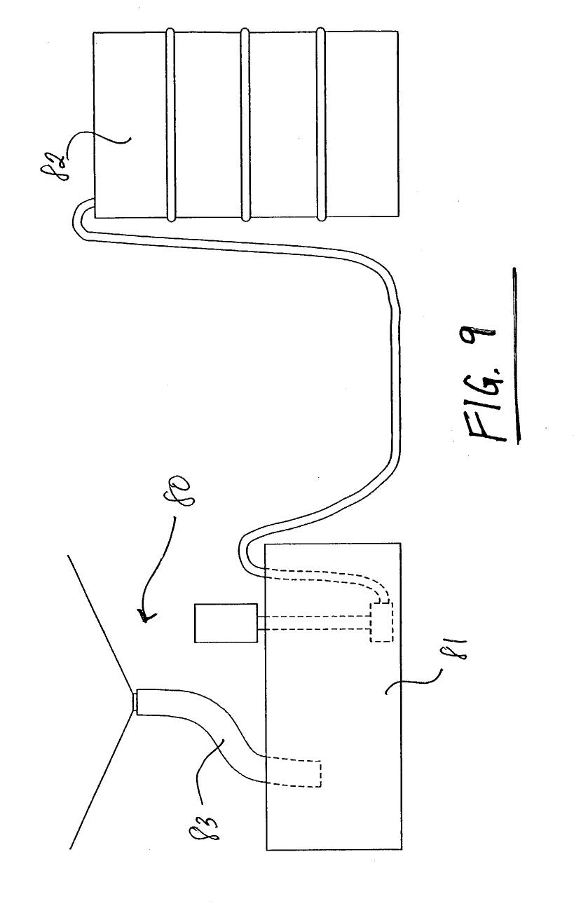

The isolation apparatus 10, in some situations, may comprise a collapsible, chemical-impervious tunnel 12 which extends between the site proximal to contamination and the washing chamber 20. However, a full-length isolation enclosure between the hazard site and the wash unit is not necessary so long as plain-clothes operators of the unit may be effectively shielded from exposure to harmful agents. For example, in the case of non-airborne contaminants, a sufficiently large wall erected between the operators of the washing chamber and the exposed area would likely be adequate to prevent operator contamination. The least expensive and simplest assembly which provides sufficient protection in a given instance is the generally preferred solution.

Where the preferred isolation apparatus 10 is a tunnel 12, the bore of such tunnel is preferably large enough to permit the passage of transport personnel and/or ambulatory victims in an upright position, as shown in FIG. 1. For example, a cylindrical isolation apparatus may have a diameter of approximately 7 feet. Typically, non-ambulatory victims are transported through the tunnel atop a stretcher 30 which is carried by appropriately trained and protected members of a HAZMAT response team. The isolation apparatus 10 of the preferred embodiment is preferably easily cleaned/decontaminated to facilitate storage and re-use. Alternatively, the isolation apparatus may be disposable. For example, a combustible apparatus may be incinerated after use to prevent further contamination.

At its distal end, the isolation apparatus 10 outputs to the washing chamber 20, as shown in FIG. 1. An airtight seal is required at this junction to prevent the release of harmful agents into the environment surrounding the decontamination system apparatus. This seal may be constructed of rubber or another durable material that prevents leakage. Additionally or alternatively, mechanical or pneumatic systems may be used to seal the connection. The seal operably surrounds the victim-receiving hatch 22 of the chamber.

Victims enter the washing chamber 20 through the receiving-hatch 22. The receiving-hatch is shown in FIG. 3. The wash chamber is aligned such that the axes of the chamber and the isolation apparatus 10, or containment barrier, are substantially parallel, as shown in FIG. 1. Patients preferably enter into, and are passed through, the washing chamber generally horizontally. An alternative design may be desirable to permit the passage of ambulatory patients through the washing chamber in a generally vertical position. Preferably, non-ambulatory victims are rolled into the washing chamber along a conveyor track 24. The conveyor track may be continuous, or it may comprise multiple sections which are assembled on opposite sites of the partition separating the wash chamber 20 from the isolation apparatus 10. A track assembly 24 must also be in place in similar manner beyond the exit hatch of the chamber for rolling patients out of the wash chamber. This conveyor track 24, and correspondingly, the receiving space 25 of the washing chamber 20 is preferably elevated above the ground on pedestal legs 26 to allow operators to stand upright while using the system, as is shown in FIGS. 2 and 5. Means, such as, for example, stools, ladders or stacking stepping blocks 29, may be necessary to elevate shorter operators to an appropriate working height, as is also shown in FIGS. 1, 2 and 5.

The washing chamber 20 preferably comprises a cylindrical or box-shaped receiving space 25 which is large enough to accept patients of various heights and sizes. FIG. 2 illustrates a cross-section of the washing chamber of a preferred design. The entry 22 and exit 28 hatches preferably comprise openings of the same, or similar, shape and dimensions as the cross-section of the wash chamber 20. FIG. 3 illustrates the entry 22 and exit 28 hatches in greater detail. For example, in an embodiment involving a cylindrical enclosure, a circular hatch of a similar diameter is used. Latching mechanisms and rubber seals, or other means for eliminating leakage, prohibit the passage of harmful agents between the wash chamber and the surrounding environment. In addition, the chamber is maintained at negative air pressure of about −1.0 to −1.5 inches H₂O of vacuum to prevent the release of contaminated molecules from the wash chamber 20. In this way, the only molecular exchange between the wash chamber and the surrounding environment as a result of opening the hatches introduces clean air into the chamber. Contaminated air is continually removed from the chamber by a fan or blower system 21 which preferably comprises a chemical-biological filtration system 23. Prior to release into the exterior airspace or another area, this air is filtered for chemical, biological or radiological contaminants. In some embodiments, the fan or blower system 21 pulls contaminated air from the chamber to create the preferred slightly negative pressure in the chamber relative to the outside ambient air, conducts the chemical-biological filtration, and forces the filtered air into the enclosed isolation apparatus, as is shown in FIG. 1 and 6. There may be adjustable vents or other means, preferably comprising filters, for allowing a small flow of outside ambient air into the chamber for maintaining suction for the fan or blower, while still allowing the fan or blower 21 to maintain a negative pressure inside the chamber.

As shown in FIG. 3, various functional elements are operably connected to the exterior of the washing enclosure 20. These elements include, but are not limited to: a plurality of glove ports 40, a receiving 22 and exit 28 hatch and means for collecting harmful substances that are released from the enclosure for disposal. In addition, where the enclosure is not constructed of a transparent material, viewing windows 42 are provided which enable operators to see inside the enclosure. It may be desirable, or necessary, to incorporate into the enclosure interior one or more mechanisms for defogging the viewing windows or other transparent material. For example, wipers (not pictured) or air circulation mechanisms such as blowers or ducts (also not pictured) may be installed to minimize or eliminate fogging, so that the operators may properly see the victim or object they are decontaminating. FIG. 3 also shows the conveyor track 24, which has been discussed previously, positioned generally in the lower portion of the wash chamber receiving space 25 and extending between the entry 22 and exit 28 hatches to assist in the transport of non-ambulatory victims through the chamber 20. This is the generally preferred positioning of the conveyor track within the receiving space though other alternatives are foreseeable. For example, positioning the track higher or lower within the enclosure may be desirable for some applications.

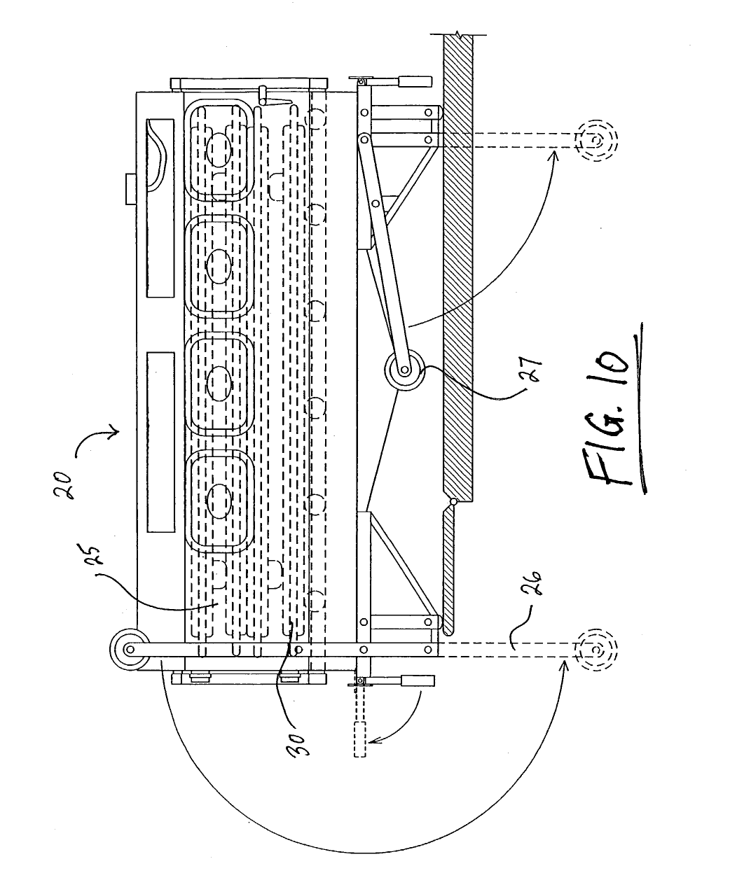

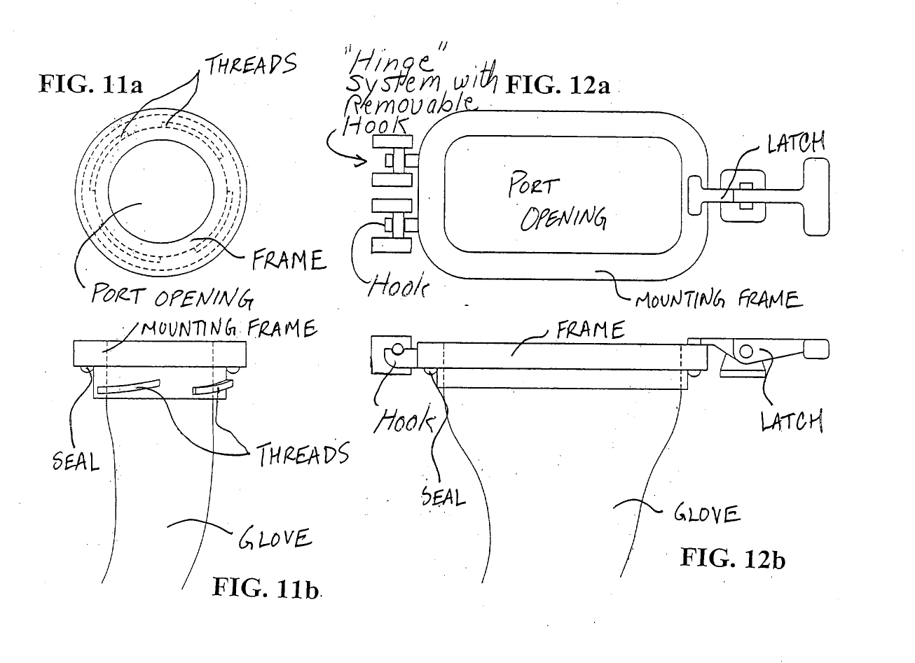

Still referring to FIG. 3, in the preferred embodiment, the glove ports 40 are mounted to the external surface of the wash chamber 20 in communication with the chamber's interior. Preferably, glove ports are provided in pairs and attached to the sides of the chamber parallel to the long axis. Gloves 41 seated within the glove ports 40, as shown in FIG. 4, permit operators to work on patients within the enclosure. The glove ports and gloves must be constructed of a durable, flexible and chemical-impermeable material such as, for example, butyl rubber. The gloves 41 are designed to accommodate most hand sizes and positioned such that every part of the victim's body may be accessed by at least one gloved hand of at least one operator. Typically, the reaching radii of operators on opposing sides of the chamber 20 overlap slightly in the region of the central axis, as illustrated in FIG. 4. This facilitates greater maneuverability and permits access to remote parts of the victim's body. Glove ports 40 of the type described above may also be mounted to the exit 22 or entry 28 hatches, as shown in FIG. 4. Preferably, such glove port assemblies are readily and quickly removed, replaced or interchanged, reinstalled and sealed. For example, a glove assembly with a threaded connector may be quickly twisted into or out of a threaded port with seal(s) that are built into the walls or hatches of the washing chamber. Alternatively, a bayonet style “stab-in and twist” system with a gasket or flexible flange may be used to create the desired quick-connect and quick-disconnect seal. An embodiment of a “stab-in and twist” system with interrupted threads is shown in FIGS. 11a & 11b. Other quick-connect and quick-disconnect assembly with seal(s), such as that shown in FIGS. 12a & 12b, may be used so long as any interruption in the decontamination process resulting from such interchange is minimal, even during prolonged procedures with many victims. Negative pressure within the chamber allows these glove/port devices to be quickly interchanged without releasing contaminants into the surrounding area.

FIG. 5 shows several operators in the process of decontaminating a patient. Decontamination procedures which are performed manually by the operators include, but are not limited to: hair removal, clothing removal, body manipulation and scrubbing. Consumable supplies such as, for example, scissors, brushes, cloths and sponges, which are required to perform these operations, may be passed into the wash unit 20 between patients. Contaminated consumables will be disposed of through the waste disposal systems as will be described in further detail below.

FIG. 6 illustrates the containment of a non-ambulatory patient within the washing chamber 20. While inside the chamber, the patient is provided with clean, breathable air via an oxygen mask 50. The oxygen mask is sealed around the patient's nose and mouth upon entry into the chamber to prevent complications associated with, for example, oxygen-deprivation and/or inhalation or ingestion of contaminants, water and/or soap. Also, goggles 52 are sealed around the patient's eyes to protect them from exposure to damaging contaminants, cleansing solutions and high-intensity sprays. Where necessary, eye-washing processes and/or systems may be implemented to safely cleanse patients' eyes.

The interior of the washing chamber 20 is outfitted with pre-positioned spray nozzles 60 in an array that is capable of completely covering the interior with liquid spray. For example, several nozzles are mounted to the upper interior surface of the washing chamber, as shown in FIG. 6. Other nozzles may be mounted to the sides, or possibly the ends of the chamber so long as operation of the glove ports 40 and hatches 22 and 28 is not inhibited. Hand-operated spray-lines 62 may also be provided to permit further, operator-controlled coverage of the interior space and the patient.

In addition, the patient carrier 30 may be adapted to allow fluid passage. For example, the carrier may include an array of holes 31 or apertures through which fluid sprays may pass to contact and decontaminate the underside of a patient, as shown in FIG. 7. This design may also facilitate more effective fluid draining at the culmination of the washing procedure. Preferably, the nozzles 60 and spray-lines 62 are connected to a foot-pedal (68) activated valve assembly 70 which is positioned beneath the unit, as shown in FIG. 8. The hand-held spray-lines 62 may be controlled by a valve assembly which is situated on the line, or line-nozzle. For example, a spray nozzle mounted to the end of the line may include a trigger-like valve assembly at the point where the nozzle is grasped to maneuver the spray-line. An example of a preferred valve assembly 70 is shown in FIG. 8. The valve assembly will deliver metered solutions of plain water, water-soap, water-bleach or other liquids or foams 69, 69′ to the spray nozzles 60 and/or hand-operated spray lines 62 at the demand of the operator(s). Water is supplied to the decontamination system 1 via the inlet supply line 71. The decontamination system may be adapted to connect to a water source with a typical garden hose or the supply line 71 may draw water from a hydrant, interior or exterior faucet, mobile holding tank, fire engine reserve tank or the like. Dry decontaminates are preferably delivered to the interior of the washing chamber 20 through the sealed hatches 22 and 28 between patients, although other means may be foreseen by one skilled in the art after reading this Description and seeing the Drawings.

A disposal system 80 facilitates the collection of contaminated liquid waste products and prevents the release of harmful substances into the environment surrounding the decontamination system. Liquid waste may drain by gravity into a trough and through a hose 83 (FIGS. 1, 5, 6, and 9) out of the bottom of the chamber 20 into a temporary catch-tank 81 (FIG. 9) or bladder. From the catch-tank 81 or bladder, the waste liquid may be pumped into a barrel 82, another container, or a container truck, for subsequent transfer and disposal, as shown in FIG. 9. Solid waste will be deposited into a bag 85, garbage bag, or other sealing container, through a sealed hatch 84 that is reachable for opening by the operator's gloved hands from the inside of the chamber. The bag 85 may be removably attached to the bottom of the hatch as shown in FIG. 6, so that it may be detached and sealed after being filled with solid waste, and then transported for disposal at a safe location. Solid wastes may include, for example, contaminated clothing, hair or fecal matter. Solid wastes are directed into the collection bags 85 by the operators working via the sealed glove ports to open the sealed hatch 84 and insert or drop the waste into the bag. These waste disposal systems function with the washing chamber and the isolation apparatus to provide a complete, integral decontamination unit. This way, a single decontamination system may be sufficient for mass decontamination at the site of exposure.

After the necessary decontamination procedures are completed, the patient is rolled out of the washing chamber 20 through the exit hatch 28. The patient is then received into an exit space 90, such as the one shown in FIG. 1, by one or several operators. This exit space may be enclosed. Once in the exit space, the patient is preferably covered with, for example, a sheet, towel or blanket for warmth and/or privacy. From here, the patient may be transported to a hospital or other treatment facility for further care.

For storing and/or transporting the decontamination system apparatus, the component parts of the system may be disassembled. Uncontaminated supplies and equipment may be stored within the receiving space 25 of the washing chamber 20, as shown in FIG. 10. For example, clean patient carriers 30 may be stacked atop one another and inserted into the chamber. Conveyor track assemblies 24 may be taken apart and stored in the enclosure as well.

In operation, the pedestal leg supports 26 that elevate the chamber must be firmly joined or locked to the washing chamber 20 to provide sufficiently stable support. However, for improved storage, these legs may be removable or collapsible. In a preferred embodiment, the pedestal legs are fixedly attached to the chamber, but collapsible, as shown in FIG. 10. For any style pedestal legs, wheels 27 may be included to facilitate rolling of the washing chamber apparatus and to simplify transport. In this case, a wheel braking system may be necessary to stabilize the apparatus while in use.

Although this invention has been described above with reference to particular means, materials and embodiments, it is to be understood that the invention is not limited to these disclosed particulars, but extends instead to all equivalents within the scope of the following claims.

I claim:

1. A decontamination system for use at or near the site of chemical, biological, radiological or other harmful exposure or at or near medical treatment or triage sites, the system comprising:
a chamber, adapted to receive a contaminated member of a group consisting of humans, animals and objects, the chamber containing, in its interior, equipment adapted to decontaminate said member, and the chamber further incorporating a plurality of ports for hands of one or more operator, which are in mechanical communication with the interior of the chamber and adapted to permit the operation of said equipment by said hands;
an isolation apparatus adapted to receive said member as the member approaches the chamber to prevent the spread of contaminants from said member into an uncontaminated space external and adjacent to said chamber; and
at least one system for collecting wastes from the group consisting of liquid, aerosol, and solid wastes for disposal;
wherein the isolation apparatus, at least one system for collecting waste, and chamber are operably connected to function as an single, integral decontamination unit.

2. A system as in claim 1, further comprising an air removal system in communication with the interior of said chamber and adapted to maintain air pressure in said interior that is negative relative to ambient air in said uncontaminated space.

3. A decontamination system according to claim 1 wherein the chamber is adapted to receive a contaminated member of said group from the isolation apparatus; cleanse said member; and output the cleansed member into said uncontaminated space external and adjacent to the chamber.

4. A decontamination system according to claim 1, adapted for use with said members that are non-ambulatory humans and non-ambulatory animals.

5. A decontamination system according to claim 1, further comprising a transport mechanism adapted to carry said members through the isolation apparatus and chamber.

6. A decontamination system according to claim 1 wherein the isolation apparatus is an elongated tunnel adapted to separate system operators from a source of contamination.

7. The system of claim 6 wherein the tunnel is selected from a group consisting of a washable tunnel, a collapsible tunnel, and a disposable tunnel.

8. The decontamination system of claim 1 wherein the isolation apparatus is a wall generally perpendicular to the longitudinal axis of the chamber.

9. The system of claim 8 wherein the wall is selected from the group consisting of a washable wall, a collapsible wall, and a disposable wall.

10. The system of claim 1, wherein said ports comprises flexible barriers between the interior of the chamber and the uncontaminated space so that the ports are adapted for use by operators not dressed in personal protective equipment.

11. The system according to claim 1 comprising a solids disposal hatch having an actuation system adapted to be actuated from inside the chamber by an operator acting through at least one of said ports.

12. The system according to claim 10 wherein the flexible barriers comprise flexible gloves seated within the ports.

13. The system according to claim 12 wherein said flexible gloves are quick-connectable and quick-releasable from said ports by means of a threaded connection.

14. The system according to claim 12 wherein said flexible gloves are quick-connectable and quick-releasable from said ports by means of a bayonet connection.

15. The system according to claim 1 wherein the chamber includes spray nozzles and lines adapted to dispense liquid sprays to the interior of the chamber.

16. A system as in claim 15 wherein said spray lines are located within the chamber and operated from outside the chamber by an operator reaching through one of said ports.

17. A decontamination system for use at or near the site of chemical, biological, radiological or other harmful exposure or at or near medical treatment or triage sites, the system comprising:
a chamber adapted to receive a human within its interior space; and
a transport system comprising a generally horizontal platform adapted to support a human, animal or object, the transport mechanism being further adapted to pass the platform and its contents through the chamber, and decontamination equipment within said chamber directed at said platform when the platform is within the chamber.

18. A decontamination system according to claim 17 wherein the platform is a stretcher and the transport system comprises a series of generally parallel and horizontal roller components.

19. A decontamination system as in claim 17 including an oxygen or fresh air supply mechanism adapted to deliver oxygen or fresh air to a human or animal on said platform.

20. A decontamination system as in claim 17, further comprising an air removal system in communication with an interior space of said chamber adapted to maintain a negative air pressure in the chamber relative to ambient air outside said chamber.

21. A decontamination system according to claim 17 further comprising ports with gloves, wherein said gloves are quick-connectable and quick-releasable from said ports by means of a threaded connection.

22. A decontamination system according to claim 17 further comprising ports with gloves, wherein said flexible gloves are quick-connectable and quick-releasable from said ports by means of a bayonet connection.

23. A decontamination system according to claim 17 wherein the chamber further comprises a solid waste disposal system situated below the transport system components and the platform.

24. A decontamination system according to claim 17 wherein the chamber further comprises a liquid waste disposal system situated below the transport system components and the platform.

25. A decontamination system for use at or near the site of chemical, biological, radiological or other harmful exposure or at or near medical treatment or triage sites, the system comprising:
an isolation apparatus adapted to separate contaminated humans, animals or objects from uncontaminated humans, animals or objects external to and near the decontamination system apparatus;
a chamber operatively connected to the isolation apparatus adapted to receive said contaminated human, animal or object, wherein the chamber includes equipment adapted to decontaminate said human, animal or object, and wherein the chamber further includes a plurality of ports for hands of operator(s) positioned along the exterior surface of the chamber, the ports being in mechanical communication with the interior of the chamber and adapted to permit the operation of said equipment by said hands; and
a transport mechanism adapted to pass said contaminated human, animal or object through the decontamination system apparatus comprising a series of generally horizontal and parallel roller components and a platform adapted to support said human, animal or object atop the roller components;
a liquid and solid wastes disposal system for collecting waste products for disposal; and
a system for removing air from the interior of the chamber adapted to create a negative pressure in the chamber relative to the exterior of the chamber.

26. A decontamination system according to claim 25 wherein the platform is a stretcher.

27. A decontamination system according to claim 25 wherein flexible gloves are seated within at least one of said ports.

28. A decontamination system according to claim 25 including an oxygen or fresh air supply mechanism adapted to deliver oxygen or fresh air to a human or animal within the chamber.

29. A decontamination system of claim 25 wherein said equipment comprises spray nozzles and spray lines adapted to dispense liquid cleaning solutions to the interior of the chamber.

30. A decontamination system of claim 29 wherein said spray lines are adapted to be maneuvered inside the chamber by an operator reaching through a port.

31. A method of decontaminating a human, animal or object exposed to chemical, biological, radiological or other harmful agents comprising:
providing a chamber for receiving a contaminated human, animal or object, having gloved ports along its exterior and decontamination equipment within its interior;
providing an apparatus for separating operators of the chamber from sources of contamination as said contaminated human, animal or object approaches the chamber, so that said operators are not dressed in personal protective equipment;
passing said human, animal or object into said chamber;
decontaminating said human, animal or object by operating said decontamination equipment by said operators contacting said human, animal or object and said equipment via said gloved ports in said chamber; and
passing said human, animal or object out of said chamber into an uncontaminated space after decontamination.

32. The method of claim 31 wherein said decontamination equipment comprises sprayers.

33. The method of claim 31 wherein said passing includes tansporting the contaminated human, animal or object on a horizontal platform.

34. The method of claim 33 further comprising said platform being rolled atop a plurality of horizontal and generally parallel roller components within the chamber.

35. The method of claim 31 further comprising operating a sealed hatch located on the chamber via said glove ports to eject waste products from the chamber.

36. The method of claim 31 further comprising maintaining a negative pressure inside the chamber relative to outside the chamber by removing air from the chamber with a fan or blower system.

37. The method of claim 31 further comprising filtering said air removed from the chamber.
